(19) United States
(12) Patent Application Publication
(10) Pub. No.: US 2005/0137661 A1
(43) Pub. Date: Jun. 23, 2005

(54) Method and system of treatment of cardiac arrhythmias using 4D imaging

(76) Inventor: Jasbir S. Sra, Pewaukee, WI (US)

Correspondence Address: JANSSON, SHUPE & MUNGER, LTD, 245 MAIN STREET, RACINE, WI, 53403, US

(21) Appl. No.: 11/016,232

(22) Filed: Dec. 17, 2004

Related U.S. Application Data
(60) Provisional application No. 60/531,295, filed on Dec. 19, 2003.
(60) Provisional application No. 60/531,293, filed on Dec. 19, 2003.

(51) Int. Cl. A61F 7/00

(52) U.S. Cl. 607/96

(57) ABSTRACT

A method is provided for treating a heart arrhythmia having the steps of obtaining cardiac digital data from a medical imaging system utilizing an ECG gated protocol; generating a series of 3D images of a cardiac chamber and its surrounding structures, preferably the left atrium and pulmonary veins, from this cardiac digital data at select ECG trigger points that correspond to different phases of the cardiac cycle; registering these 3D images with an interventional system; acquiring ECG signals from the patient in real-time; transmitting these ECG signals to the interventional system; synchronizing the registered 3D images with trigger points on the transmitted ECG signals to generate a 4D image; visualizing this 4D image upon the interventional system in real-time; visualizing a catheter over the 4D image upon the interventional system; navigating the catheter within the cardiac chamber utilizing the 4D image; and using the catheter to treat the cardiac chamber, preferably with ablation.

### RELATED APPLICATION

This application claims the benefit of U.S. Provisional Applications Nos. 60/531,295 and 60/531,293, each filed on Dec. 19, 2003 and the contents of each are incorporated by reference herein in its entirety.

### FIELD OF THE INVENTION

This invention relates generally to methods and systems for treatment of atrial fibrillation and other cardiac arrhythmias and, in particular, to methods and systems utilizing 3D digital images for cardiac interventional procedures in such treatment and for the planning of such procedures.

### BACKGROUND OF THE INVENTION

Heart rhythm problems or cardiac arrhythmias are a major cause of mortality and morbidity. An example of different rhythm problems encountered in clinical practice include atrial fibrillation (AF), cardiac arrest or sudden cardiac death (SCD) due to ventricular tachycardia/ventricular fibrillation (VT/VF), atrial flutter and other forms of atrial and ventricular arrhythmias. During the past 20 years, cardiac electrophysiology has evolved into a clinical tool to diagnose these cardiac arrhythmias. During electrophysiology studies multipolar catheters are positioned inside the heart and electrical recordings are made from the different chambers of the heart. Arrhythmias can be initiated in the laboratory using programmed electrical stimulation. Careful study of surface ECG and data from intracavitary electrograms is used conventionally to treat these arrhythmias. However, essential to the management of any cardiac rhythm problem is a thorough understanding of the mechanism of its initiation and sustenance. A short discussion of these arrhythmias and their mechanisms is thus useful in understanding the complexity of the problem and how the invention presented will help facilitate the treatment of these arrhythmias.

Atrial fibrillation is dysrhythmia of the atria or the upper chambers of the heart in which the atria stop contracting as they begin to fibrillate or quiver. Atrial fibrillation is the most common sustained arrhythmia encountered in clinical practice and, recent data suggests, the most common arrhythmia related cause of hospital admissions. Estimates indicate that 2.2 million people in the United States alone have AF and that 160,000 new cases are diagnosed every year. Patients with AF have a high incidence of such complications as stroke, and heart failure and bear an ominous prognosis of higher overall and cardiovascular mortality.

Recently it has been shown that premature atrial contractions can act as triggers and initiate paroxysms of AF. These premature ectopic beats have been shown to originate predominantly in the pulmonary veins. Inability to reproducibly identify the precise location of these trigger sites limits catheter ablation of trigger sites of AF. Because of the critical role of the pulmonary veins in the generation of AF, and as infrequent and nonreproducible premature atrial contractions limit the utility of trigger site ablation, a variety of surgical and nonsurgical catheter ablation techniques have been used to isolate the pulmonary veins from the left atrium. Intraoperative complete isolation of the pulmonary veins using various energy sources in patients undergoing open heart surgery has led to successful termination of AF in over 80% of patients. Trying to replicate this procedure nonsurgically is lengthy and labor intensive. Usually two catheters are positioned inside the left atrium guided by fluoroscopy. As the left atrium-pulmonary vein junction cannot be seen, the catheters are swept around and only electrical signals are used to guide the catheters to locations to which heat is delivered. As true anatomy is not visualized, the success rate of this procedure is low and only a limited number of patients qualify for this procedure. A method, based on an anatomically based model using transvenous catheters, which enables the rapid encircling of the pulmonary veins with a series of accurately placed radio-frequency lesions or lesions using other forms of energy such as microwave, cryoablation, laser and others, would offer a less invasive alternative to surgery.

Sudden cardiac death (SCD) is defined as an unexpected natural death from cardiac causes within a short period of time. Most such deaths are caused by VT/VF. It is estimated that SCD accounts for approximately 300,000 cardiac deaths in the United States alone each year. SCD is the most common and often the first manifestation of coronary artery disease and may be responsible for approximately 50% of deaths from cardiovascular disease in the United States. The most commonly encountered form of VT typically originates in the vicinity of a healed myocardial infarction. The mechanism of VT is reentry associated with myocardial scarring. However, these reentrant circuits are quite broad because of the nature of the scarring. The success rate of VT ablation would increase considerably if it were possible to interrupt these broad reentrant circuits using lesions transecting these circuits. This would require: 1) precise identification of the margins of scarring, 2) the ability to identify and return precisely to areas of interest and, 3) the ability to visualize the lesion lines created. A method allowing precise anatomical delineation of the ventricle would make this possible.

Several other arrhythmias such as atrial flutter, atrial tachycardia, and tachycardia involving accessory connections between the atria and ventricles are also extremely common and cause significant morbidity and some risk of higher mortality. The mechanism of atrial flutter has also been identified. Ablation between the tricuspid annulus and inferior vena cava, forming an anatomical barrier around the flutter circuit, can terminate atrial flutter. Precise identification of this anatomy would thus help significantly. Similarly, precise location and identification of areas such as the crista terminalis in the right atrium, which is a common source of atrial tachycardia, would be useful.

A number of new techniques are aimed at improving the resolution and acquisition of cardiac activation maps during electrophysiology studies. Although helpful in many instances, inability to accurately relate electrophysiologic information to a specific anatomical location in the heart limits their ability to treat complex arrhythmias such as AF, VT and other arrhythmias. The image created is not an exact replication of the anatomy of specific locations in the cardiac chamber. Degree of resolution of the image is totally operator-dependent and limited by the time available to acquire data points.

A number of modalities exist for medical diagnostic imaging. The most common ones for delineating anatomy include computer tomography (CT), magnetic resonance imaging (MRI) and x-ray systems. CT imaging is a fast and accurate way to delineate the anatomy of any organ. The ability to collect volumes of data at short acquisition times allows for 3-D reconstruction of images resulting in true depictions and more understandable anatomic images.

The role of CT in the management of cardiac rhythm problems has been, however, insignificant for several reasons which include motion artifacts in a beating structure such as the heart, and the inability to delineate the origin and propagation of electrical impulses. Use of cardiac gating allows acquisition of consecutive axial images from the same phase of a cardiac cycle. This will allow elimination of motion artifacts. Surface rendering techniques make it possible to view both endocardial (inside) and epicardial (outside) views of any chamber.

Although the 3D images of the different cardiac chambers could be created by the modalities mentioned before these images even if they can be registered on an interventional system are still and do not replicate the motion of the heart real-time. It is thus not possible to assess the different aspects of the motion of the heart such as systole (contraction) or diastole (relaxation). This is critical if the mapping and ablation catheters need to be navigated to the appropriate sites for successful results during the intervention procedure an to avoid complications such as perforation of the heart during the procedure as the exact orientation and location of the catheter is not possible in a still image.

The drawbacks discussed above and deficiencies of the prior art are overcome with a method and system of 4D imaging where the reconstructed 3D images are seen in real-time over different phases of the cardiac cycle.

### SUMMARY OF THE INVENTION

One aspect of this invention provides a method for treatment of a heart arrhythmia, preferably atrial fibrillation, in a patient using 4D imaging. The method has the steps of (1) obtaining cardiac digital data from a medical imaging system utilizing an electrocardiogram (ECG) gated protocol; (2) generating a series of three-dimensional (3D) images of a cardiac chamber and its surrounding structures from this cardiac digital data, the data being gated at select ECG trigger points having correspondence with different phases of the cardiac cycle; (3) registering these 3D images with an interventional system; (4) acquiring ECG signals from the patient in real-time; (5) transmitting these ECG signals to the interventional system; (6) synchronizing the registered 3D images with certain corresponding trigger points on the transmitted ECG signals such that a 4D image covering the different phases of the cardiac cycle is generated; (7) visualizing this 4D image upon the interventional system in real-time; (8) visualizing a catheter over the 4D image also upon the interventional system; (9) navigating the catheter within the cardiac chamber utilizing the 4D image; and then (10) using the catheter to treat the cardiac chamber, preferably with ablation.

In a desirable embodiment, the medical imaging system is a computer tomography (CT) system. Also preferred is where the imaging system is a magnetic resonance imaging (MRI) system or one utilizing ultrasound. Most desirable is where the method also includes the step of visualizing the 4D image over a computer workstation of the interventional system.

One very preferred embodiment finds the 3D images are of the left atrium and pulmonary veins. More preferred is where the catheter is one adapted for mapping and ablation. Most preferred is where the step of generating 3D images from the cardiac digital data uses a protocol optimized for 3D imaging of the left atrium and pulmonary veins.

Certain exemplary embodiments are where the interventional system is a fluoroscopic system. Also highly desired are embodiments having the additional step of continuously updating and adjusting the synchronization of the registered 3D images with the trigger points on the transmitted ECG signals during an interventional procedure.

Another aspect of this invention finds a system for providing treatment of a heart arrhythmia in a patient. This system has a medical imaging system for obtaining cardiac digital data utilizing an electrocardiogram (ECG) gated protocol; an image generation system for generating a series of three-dimensional (3D) images of a cardiac chamber and surrounding structures from the cardiac digital data at select ECG trigger points that correspond to different phases of the cardiac cycle; an ECG monitor for acquiring ECG signals from the patient in real-time and for transmitting these ECG signals to an interventional system; a workstation for registering the 3D images with an interventional system and for then synchronizing these registered 3D images with trigger points on the transmitted ECG signals to generate a 4D image that is visualized upon the interventional system in real-time; and a catheter apparatus for treating heart tissue within the cardiac chamber at select locations, the catheter apparatus having a catheter visualized upon the interventional system over the 4D image.

A preferred embodiment is where the medical imaging system is a computer tomography (CT) system. Also preferred is where the 3D images are of the left atrium and pulmonary veins. Most preferred is when the catheter is adapted for mapping and ablation. Highly preferred cases find that the image generation system generates 3D images from the cardiac digital data utilizing a protocol optimized for 3D imaging of the left atrium and pulmonary veins.

In certain desirable embodiments, the interventional system is a fluoroscopic system. Most desirable is where the workstation continuously updates and adjusts the synchronization of the registered 3D images with the trigger points on the transmitted ECG signals during an interventional procedure.

In another aspect of this invention, a method is provided for planning treatment of a patient's heart arrhythmia. This method includes the steps of (1) obtaining cardiac digital data from a medical imaging system utilizing an electrocardiogram (ECG) gated protocol; (2) generating a series of three-dimensional (3D) images of a cardiac chamber and surrounding structures from the cardiac digital data at select ECG trigger points corresponding with different phases of the cardiac cycle; (3) registering the 3D images with an interventional system; (4) acquiring ECG signals from the patient in real-time; (5) transmitting the ECG signals to the interventional system; (6) synchronizing the registered 3D images with trigger points on the transmitted ECG signals to generate a 4D image; and (7) visualizing the 4D image upon the interventional system in real-time.

Yet another aspect of this invention finds a system for planning treatment of a heart arrhythmia. The system comprises a medical imaging system for obtaining cardiac digital data utilizing an electrocardiogram (ECG) gated protocol; an image generation system for generating a series of three-dimensional (3D) images of a cardiac chamber and its surrounding structures from the cardiac digital data at select ECG trigger points corresponding to different phases of the cardiac cycle; an ECG monitor for acquiring ECG signals from the patient in real-time and for transmitting the ECG signals to an interventional system; and a workstation for registering the 3D images with an interventional system and for synchronizing the registered 3D images with trigger points on the transmitted ECG signals to generate a 4D image that is visualized upon the interventional system in real-time.

### BRIEF DESCRIPTION OF THE DRAWINGS

FIG. 1 is a schematic overview of a system for treatment of a heart arrhythmia in accordance with this invention.

FIG. 2A depicts 3D cardiac images of the left atrium.

FIG. 2B illustrates localization of a standard mapping and ablation catheter in real-time over an endocardial view of the left atrium registered upon an interventional system.

FIG. 3 is a flow diagram of a method for treatment of atrial fibrillation and other cardiac arrhythmias in accordance with this invention.

FIG. 4 is an example of 3D images of the left ventricle that are depicted as being synchronized to the systole (contraction) and diastole (relaxation) phases of the cardiac cycle.

### DETAILED DESCRIPTION OF PREFERRED EMBODIMENTS

The drawings illustrate embodiments of a system and method for treating heart arrhythmia in a patient using 4D imaging in accordance with this invention. The embodiments shown enable an electrophysiologist, cardiologist and/or surgeon to plan in advance and to later perform an interventional procedure such as atrial fibrillation ablation in a manner that makes the procedure simpler and more efficacious while decreasing the risk of complications.

Using imaging systems known in the art, 3D images are obtained of a cardiac chamber such as the left atrium and its adjacent pulmonary veins. These images include detailed 3D models and endocardial views (i.e., navigator or views from the inside) of the chamber. These images are then registered and synchronized with real-time cardiac motion on an interventional system such as a fluoroscopic system to generate a 4D image. In this manner, detailed 3D images acquired at different phases of the cardiac cycle prior to an interventional procedure constitute displacement profiles of the cardiac chamber that can be visualized sequentially in real-time during the procedure.

In addition, a mapping/ablation catheter may be seen over these images so that the practitioner can navigate the catheter to strategic locations within the left atrium such as the left atrial-pulmonary vein junctions in a manner where the orientation and location of the catheter is better understood to avoid complications such as perforation of the heart during the procedure.

Although the embodiments illustrated are described in the context of a CT imaging system, it will be appreciated that other imaging systems known in the art, such as MRI and ultrasound, are also contemplated with regard to obtaining cardiac digital data for generating 3D images of the heart. Similarly, although the interventional system is described in the context of fluoroscopy and an associated computer work station, other interventional systems are also contemplated. In addition to viewing the left atrium, the anatomy of other cardiac chambers can also be imaged, registered and visualized.

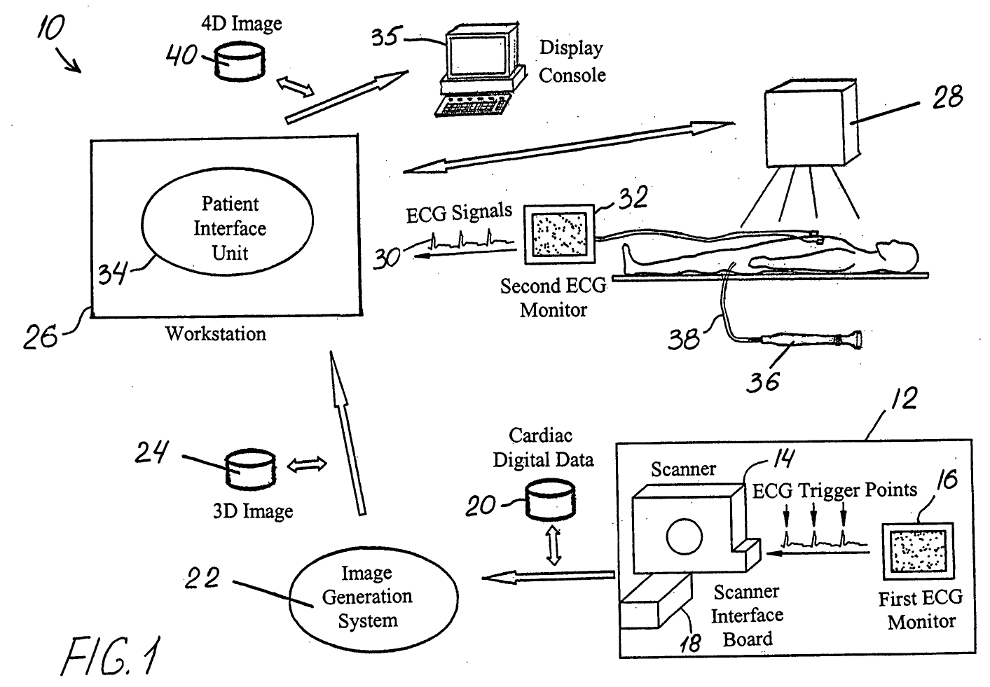

There is shown in FIG. 1 an schematic overview of an exemplary system 10 for treatment of a heart arrhythmia in a patient in accordance with this invention. System 10 includes CT imaging system 12 having a scanner 14 and a first ECG monitor 16 that outputs ECG trigger points corresponding with different phases of the cardiac cycle to scanner 14 through a scanner interface board 18 utilizing a ECG gated protocol. A suitable example of scanner interface board 18 is a Gantry interface board. Scanner 14 therefore utilizes ECG-gated acquisition to image the heart at different phases of the cardiac cycle such as when the heart is free of motion and its diastolic phase, as well as in multiple phases of systole and early diastole.

Scanner 14 outputs cardiac digital data 20, including ECG signal time-stamps associated with such data generated by the gating protocol, to image generation system 22. Image generation is performed using one or more optimized 3D protocols for automated image segmentation of the cardiac digital data for the left atrium and such surrounding structures as the pulmonary veins. A series of gated 3D images 24 corresponding to the selected ECG trigger points are thus generated having quantitative features of the left atrium such as its contour, orientation and thickness as well as providing endocardial or “immersible” views of the ostial areas between the left atrium and the pulmonary veins. 3D images 24 may be in any one of several formats, including but not limited to: a wire mess geometric model, a set of surface contours, a segmented volume of binary images, and a DICOM (Digital Imaging and Communications in Medicine) object using the radiation therapy DICOM object standard.

3D images 24 are exported from image generation system 22 and registered with workstation 26 of fluoroscopic system 28. ECG signals 30 are generated by second ECG monitor 32 and transmitted by ECG monitor 32 to workstation 26. ECG signals 30 contain data referable to an ECG being performed on the patient in real-time using ECG monitor 32 during the interventional procedure.

Workstation 26 includes patient interface unit 34 that places ECG signals 30 in communication with 3D images 24. Interface unit 34 is a processing unit that analyzes ECG signals 30 and synchronizes 3D images 24 with the real-time cardiac cycle of the patient by recognizing the ECG signal time-stamps on the images and matching them with the corresponding points on the real-time ECG. A zero time differential between these two values is calculated by workstation 26 to enhance synchronization. In this manner, 4D imaging 40 of the left atrium is visualized on the interventional system at a display console 35.

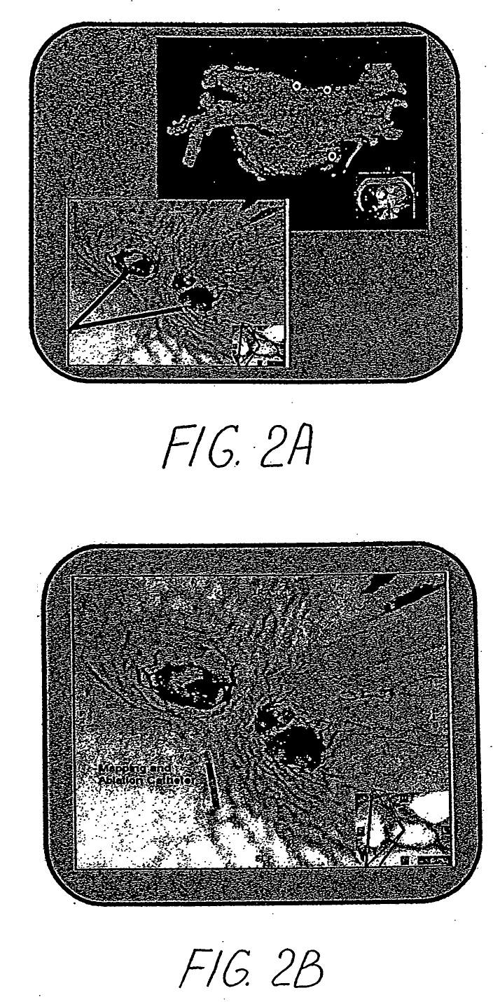

A detailed 3D model of the left atrium and the pulmonary veins, including endocardial or inside views, is seen in FIG. 2A. The distance and orientation of the pulmonary veins and other strategic areas can be calculated in advance from such images. 3D images of this type are used to generate 4D imaging in accordance with this invention, thereby creating a roadmap for use during an ablation procedure.

During the interventional procedure, a catheter apparatus 36 having a mapping/ablation catheter 38 is delivered to the left atrium typically using a transeptal catheterization. Catheter 38 is continuously localized on fluoroscopic system 28 whereby catheter 38 is visualized over 4D image 40. Having catheter 38 seen over 4D image 40 in real-time enables the practitioner to safely and accurately navigate catheter 38 in real-time to the appropriate sites within the left atrium and its surrounding structures where radio-frequency energy can be delivered to ablate heart tissue in treatment of atrial fibrillation. FIG. 2B illustrates localization of a standard mapping and ablation catheter over an endocardial view of the left atrium registered upon an interventional system.

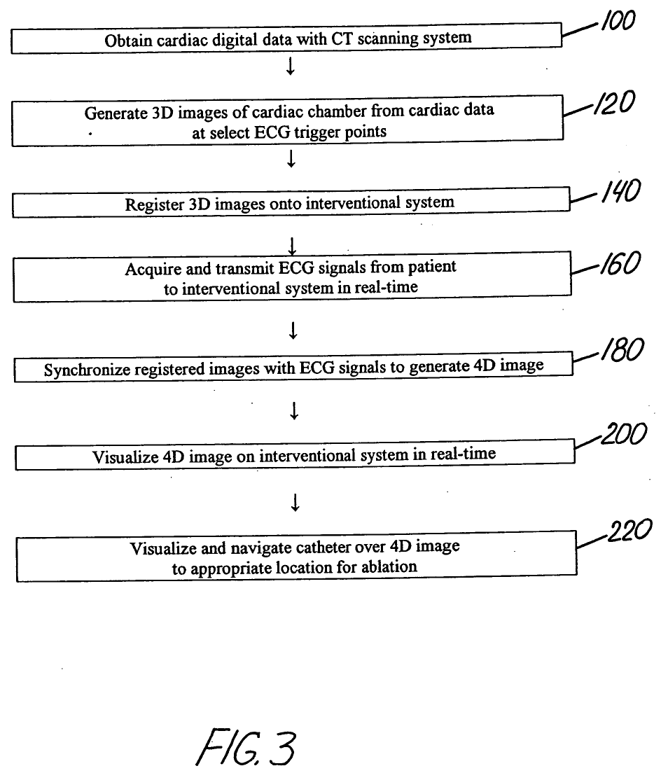

FIG. 3 illustrates a schematic overview of the method for treating a heart arrhythmia using 4D imaging in accordance with this invention. As shown in step 100, the CT scanning system is used to obtain cardiac digital data. The CT imaging system is automated to acquire a continuous sequence of data of the patient's heart. A shorter scanning time using a faster scanner and synchronization of the CT scanning with a gated ECG signal of the patient at select trigger points reduces the motion artifacts in a beating organ like the heart and provides displacement profiles of the heart at different phases of the cardiac cycle. The ability to collect a volume of data in a short acquisition time allows reconstruction of cardiac images in more accurate geometric depictions, thereby making them easier to understand.

In step 120, the data-set acquired by the CT imaging system is segmented and a series of 3D images of the left atrium and surrounding pulmonary veins is generated using protocols optimized for those structures. The 3D images identify and visualize the desired views of the left atrium at select points within the cardiac cycle.

As shown in step 140, the 3D images are then exported and registered with an interventional system such as one using fluoroscopy. The transfer of 3D images, including 3D model and navigator views, can occur in several formats such as DICOM format or object and geometric wire mesh model.

The registration method transforms the coordinates in the CT images into the coordinates in the fluoroscopic system. Information acquired by the CT scanning system will in this manner be integrated in real-time with imaging of the left atrium by the fluoroscopic system. Once these coordinates are locked in between the 3D images and the fluoroscopic views, the 3D models and navigator views can be seen from different perspectives on the fluoroscopic system.

At step 160, ECG signals are acquired from the patient at the time of the interventional procedure. These signals are transmitted to the interventional system and brought into communication with the 3D images through a patient interface unit. In step 180, the interface unit analyzes the ECG signals received and synchronizes these signals with the gated 3D images to generate a 4D image. Several trigger points are recognized on both the real-time ECG and the ECG time-stamped 3D images and a zero time differential between these values is calculated.

As seen at step 200, this 4D image comprising multiple views of the left atrium can then be viewed sequentially in synchronization with the various phases of the cardiac cycle in real-time on the interventional system. Preferably, the synchronization of the 3D images with the real-time ECG signals is continuously updated and adjusted during the interventional procedure.

In addition, as shown at step 220, the invention further involves the location of a mapping/ablation catheter over the fluoroscopic system and, in particular, over the registered 4D image of the left atrium and surrounding structures. The catheter is then navigated to the appropriate site within the left atrium in a less risky and efficient manner to perform the necessary ablation procedure in treatment of the patient's arrhythmia.

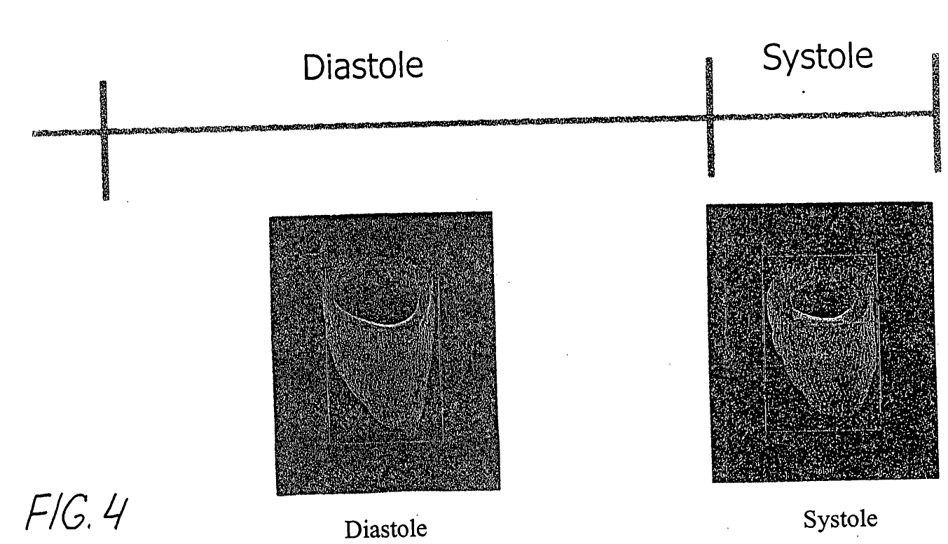

FIG. 4 is an example of 3D images depicting relaxation (diastole) and contraction (systole) of the left ventricle. The different displacement profiles are shown synchronized to a ECG signal where different trigger points are shown as small lines transecting the different phases of the cardiac cycle as shown by the horizontal line.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for treating of a heart arrhythmia in a patient using 4D imaging comprising:
obtaining cardiac digital data from a medical imaging system utilizing an electrocardiogram (ECG) gated protocol;
generating a series of three-dimensional (3D) images of a cardiac chamber and surrounding structures from the cardiac digital data at select ECG trigger points corresponding with different phases of the cardiac cycle;
registering the 3D images with an interventional system;
acquiring ECG signals from the patient in real-time;
transmitting the ECG signals to the interventional system;
synchronizing the registered 3D images with trigger points on the transmitted ECG signals to generate a 4D image;
visualizing the 4D image upon the interventional system in real-time;
visualizing a catheter over the 4D image upon the interventional system;
navigating the catheter within the cardiac chamber utilizing the 4D image; and
using the catheter to treat the cardiac chamber.

2. The method of claim 1 wherein the medical imaging system is a computer tomography (CT) system.

3. The method of claim 1 further comprising the step of visualizing the 4D image over a computer workstation of the interventional system.

4. The method of claim 1 wherein the 3D images are of the left atrium and pulmonary veins.

5. The method of claim 4 wherein the catheter is adapted for mapping and ablation.

6. The method of claim 5 wherein generating 3D images from the cardiac digital data comprises using a protocol optimized for 3D imaging of the left atrium and pulmonary veins.

7. The method of claim 1 wherein the interventional system is a fluoroscopic system.

8. The method of claim 1 further comprising the step of continuously updating and adjusting the synchronization of the registered 3D images with the trigger points on the transmitted ECG signals during an interventional procedure.

9. A system for treating of a heart arrhythmia in a patient using 4D imaging comprising:
a medical imaging system for obtaining cardiac digital data utilizing an electrocardiogram (ECG) gated protocol;
an image generation system for generating a series of three-dimensional (3D) images of a cardiac chamber and surrounding structures from the cardiac digital data at select ECG trigger points corresponding with different phases of the cardiac cycle;
an ECG monitor for acquiring ECG signals from the patient in real-time and for transmitting the ECG signals to an interventional system;
a workstation for registering the 3D images with the interventional system and for synchronizing the registered 3D images with trigger points on the transmitted ECG signals to generate a 4D image that is visualized upon the interventional system in real-time; and
a catheter apparatus for treating heart tissue within the cardiac chamber at select locations, the catheter apparatus having a catheter visualized over the 4D image upon the interventional system.

10. The system of claim 9 wherein the medical imaging system is a computer tomography (CT) system.

11. The system of claim 9 wherein the 3D images are of the left atrium and pulmonary veins.

12. The system of claim 11 wherein the catheter is adapted for mapping and ablation.

13. The system of claim 12 wherein the image generation system generates 3D images from the cardiac digital data utilizing a protocol optimized for 3D imaging of the left atrium and pulmonary veins.

14. The system of claim 9 wherein the interventional system is a fluoroscopic system.

15. The system of claim 9 wherein the workstation continuously updates and adjusts the synchronization of the registered 3D images with the trigger points on the transmitted ECG signals during an interventional procedure.

16. A method for planning treatment of a heart arrhythmia in a patient using 4D imaging comprising:
obtaining cardiac digital data from a medical imaging system utilizing an electrocardiogram (ECG) gated protocol;
generating a series of three-dimensional (3D) images of a cardiac chamber and surrounding structures from the cardiac digital data at select ECG trigger points corresponding with different phases of the cardiac cycle;
registering the 3D images with an interventional system;
acquiring ECG signals from the patient in real-time;
transmitting the ECG signals to the interventional system;
synchronizing the registered 3D images with trigger points on the transmitted ECG signals to generate a 4D image; and
visualizing the 4D image upon the interventional system in real-time.

17. The method of claim 16 wherein the medical imaging system is a computer tomography (CT) system.

18. The method of claim 17 wherein generating 3D images from the cardiac digital data comprises using a protocol optimized for 3D imaging of the left atrium and pulmonary veins.

19. The method of claim 18 wherein the interventional system is a fluoroscopic system.

20. A system for planning treatment of a heart arrhythmia in a patient using 4D imaging comprising:
a medical imaging system for obtaining cardiac digital data utilizing an electrocardiogram (ECG) gated protocol;
an image generation system for generating a series of three-dimensional (3D) images of a cardiac chamber and surrounding structures from the cardiac digital data at select ECG trigger points corresponding with different phases of the cardiac cycle;
an ECG monitor for acquiring ECG signals from the patient in real-time and for transmitting the ECG signals to an interventional system; and
a workstation for registering the 3D images with an interventional system and for synchronizing the registered 3D images with trigger points on the transmitted ECG signals to generate a 4D image that is visualized upon the interventional system in real-time.
